(19) United States
(12) Patent Application Publication
(10) Pub. No.: US 2005/0277839 A1
(43) Pub. Date: Dec. 15, 2005

(54) Wireless flow measurement in arterial stent

(75) Inventors: Richard A. Alderman, Freeport, IL (US); Patrick S. Gonia, Maplewood, MN (US); James Z. Liu, Rockford, IL (US)

Correspondence Address: Attorney, Intellectual Property;Honeywell International, Inc., 101 Columbia Rd., P.O. Box 2245, Morristown, NJ, 07962, US

(73) Assignee: Honeywell International, Inc.

(21) Appl. No.: 10/866,423

(22) Filed: Jun. 10, 2004

(51) Int. Cl. A61B 5/02

(52) U.S. Cl. 600/504; 600/481

(57) ABSTRACT

A blood flow sensing system is disclosed, including a sensor coupled to an antenna, such that the sensor measures a flow of blood within a blood vessel when stimulated with a short range radio frequency energy field detectable by the antenna. Such a system additionally can include a transmitter and receiver unit (i.e., a transmitter/receiver), which can transmit the short range radio frequency energy field to the antenna of the sensor. The transmitter and receiver unit can also receive data transmitted from the sensor via the antenna. Such a system additionally includes a stent integrated with sensor, wherein the stent comprises a small diameter cylinder that props open a blood vessel and wherein the stent is moveable into the blood vessel to form a rigid support for holding the blood vessel open in order to measure the flow of blood within the blood vessel.

### TECHNICAL FIELD

Embodiments are generally related to flow sensing devices and techniques. Embodiments are also related to stents, such as, for example, arterial stents utilized in medical procedures. Embodiments are also related to surface wave sensor devices and systems, including interdigital sensors.

### BACKGROUND OF THE INVENTION

Cardiac output or blood flow is one of the key indicators of the performance of the heart. Blood flow can be defined as volume of blood or fluid flow per time interval. Fluid or fluid velocity is generally a function of flow area at the measurement site. Use of blood flow measurements allows discrimination between physiologic rhythms, such as sinus tachycardia, which is caused by exercise or an emotional response, and other pathologic rhythms, such as ventricular tachycardia or ventricular fibrillation.

Cardiac arrhythmia is defined as a variation of the rhythm of the heart from normal. The cardiac heartbeat normally is initiated at the S-A node by a spontaneous depolarization of cells located there during diastole. Disorders of impulse generation include premature contractions originating in abnormal or ectopic foci in the atria or ventricles, paroxysmal supraventricular tachycardia, atrial flutter, atrial fibrillation, ventricular tachycardia and ventricular fibrillation. Ventricular arrhythmia can occur during cardiac surgery or result from myocardial infarction. Ventricular tachycardia presents a particularly serious problem because the patient, if left untreated, may progress into ventricular fibrillation.

Blood flow measurements allow discrimination between normal and pathologic rhythms by providing a correlation between the electrical activity of the heart and the mechanical pumping performance or fluid flow activity of the heart. During sinus tachycardia, an increase in heart rate will usually be accompanied by an increase in cardiac output or blood flow. During ventricular tachycardia or ventricular fibrillation, heart rate increase will be accompanied by a decrease in, or perhaps a complete absence of, cardiac output or blood flow. A number of important cardiac and clinical devices may be improved by a more accurate measure of cardiac output. The ability to measure blood flow can be applied to the following four areas: (1) automatic implantable defibrillators, (2) rate adaptive pacemakers, (3) cardiac output diagnostic instruments and (4) peripheral blood flow instruments.

Conventional methods of measuring blood flow have included blood thermal dilution, vascular flow monitoring, and injectionless thermal cardiac output. Such procedures are typically extremely invasive or can be unreliable. The ability to measure and detect blood flow is thus of key importance to maintaining proper health, before, during and following surgical procedures such as angioplasty.

Medical stents are used within the body to restore or maintain the patency of a body lumen. Blood vessels, for example, can become obstructed due to plaque or tumors that restrict the passage of blood. A stent typically has a tubular structure defining an inner channel that accommodates flow within the body lumen. A stent can be configured in the form of a small, expandable wire mesh tube. The outer walls of the stent engage the inner walls of the body lumen. Positioning of a stent within an affected area can help prevent further occlusion of the body lumen and permit continued flow.

A stent typically is deployed by percutaneous insertion of a catheter or guide wire that carries the stent. The stent ordinarily has an expandable structure. Upon delivery to the desired site, the stent can be expanded with a balloon mounted on the catheter. Alternatively, the stent may have a biased or elastic structure that is held within a sheath or other restraint in a compressed state. The stent expands voluntarily when the restraint is removed. In either case, the walls of the stent expand to engage the inner wall of the body lumen, and generally fix the stent in a desired position.

Stents can be utilized in a procedure known as “stenting,” which is a non-surgical treatment utilized is association with balloon angioplasty to treat coronary artery disease. Immediately following angioplasty, which can result in the widening of a coronary artery, the stent can be inserted into the blood vessel. The stent assists in holding open the newly treated artery, thereby alleviating the risk of the artery re-closing over time.

An example of a stent is disclosed in non-limiting U.S. Pat. No. 6,709,440, “Stent and Catheter Assembly and Method for Treating Bifurcations,” which issued to Callol et al on Mar. 23, 2004, and which is incorporated herein by reference. Another example of a stent is disclosed in non-limiting U.S. Pat. No. 6,699,280, “Multi-Section Stent,” which issued to Camrud et al on Mar. 2, 2004, and which is incorporated herein by reference. A further example of a stent is disclosed in non-limiting U.S. Pat. No. 6,695,877, “Bifurcated Stent,” which issued to Brucker et al on Feb. 24, 2004, and which is incorporated herein by reference.

Surface wave sensors can be utilized in a number of sensing applications. Examples of surface wave sensors include devices such as acoustic wave sensors, which can be utilized to detect the presence of substances, such as chemicals. An acoustic wave (e.g., SAW/SH-SAW/Love/SH-APM) device acting as a sensor can provide a highly sensitive detection mechanism due to the high sensitivity to surface loading and the low noise, which results from their intrinsic high Q factor.

Surface acoustic wave devices are typically fabricated using photolithographic techniques with comb-like interdigital transducers placed on a piezoelectric material. Surface acoustic wave devices may have either a delay line or a resonator configuration. The change of the acoustic property due to the flow can be interpreted as a delay time shift for the delay line surface acoustic wave device or a frequency shift for the resonator (SH-SAW/SAW) acoustic wave device.

Acoustic wave sensing devices often rely on the use of piezoelectric crystal resonator components, such as the type adapted for use with electronic oscillators. In a typical flow sensing application, the heat convection can change the substrate temperature, while changing the SAW device resonant frequency. With negative temperature coefficient materials such as LiNbO₃, the oscillator frequency is expected to increase with increased liquid flow rate. The principle of sensing is similar to classical anemometers.

Flow rate is an important parameter for many applications. The monitoring of liquid (e.g., blood, saline, etc.) flow rate within and/or external to a living body (e.g., human, animal, etc) can provide important information for medical research and clinical diagnosis. Such measurements can provide researchers with insights into, for example, the physiology and functioning of the heart and other human organs, thereby leading to advances in medical, nutrition and related biological arts. Blood/liquid flow rate measurements can also provide useful information regarding the safety and efficacy of pharmaceuticals and the toxicity of chemicals.

It is believed that the use of passive, wireless acoustic wave devices for blood flow rate monitoring can provide for great advances in physiological, pharmaceutical and medical applications to name a few. Surface acoustic wave sensors have the potential to provide flow sensor systems with higher sensitivity and wider dynamic ranges than the solid state flow sensor devices currently available. To date such devices have not been incorporated successfully into medical applications, particularly those involving the use of stents.

### BRIEF SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

It is, therefore, one aspect of the present invention to provide for improved blood flow sensor devices and sensing techniques.

It is another aspect of the present invention to provide for an improved surface wave flow sensor device that can be adapted for use in blood flow sensing applications.

It is yet a further aspect of the present invention to provide for an interdigital surface wave device, such as, for example, surface acoustic wave (SAW) resonator or surface acoustic wave (SAW) delay line sensing devices, which can be adapted for use in blood flow sensing applications.

It is a further aspect of the present invention to provide for a wireless blood flow sensor, which can be integrated with a stent used in medical procedures, for blood flow sensing activities thereof.

It is an additional aspect of the present invention to provide for a blood flow sensor that also measures temperature and pressure utilizing interdigital (IDT) temperature and pressure sensor elements integrated with the blood flow sensor.

The aforementioned aspects of the invention and other objectives and advantages can now be achieved as described herein A blood flow sensing system is thus disclosed, which can include a sensor coupled to an antenna, such that the sensor measures a flow of blood within a blood vessel when stimulated with a short range radio frequency energy field detectable by the antenna. Such a system additionally can include a transmitter and receiver unit (i.e., a transmitter/receiver), which can transmit the short range radio frequency energy field to the antenna of the sensor.

The transmitter and receiver unit can also receive data transmitted from the sensor via the antenna. Such a system additionally includes a stent integrated with sensor, wherein the stent comprises a small diameter cylinder that props open a blood vessel and wherein the stent is moveable into the blood vessel to form a rigid support for holding the blood vessel open in order to measure the flow of blood within the blood vessel. The stent can also be configured to include a wire mesh that supports the functionality of the antenna. The sensor itself measures heat transfer to blood within the blood vessel. The sensor can be configured, however, to incorporate pressure and temperature sensing elements. Such pressure and temperature sensing elements may be interdigital transducer components.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a perspective view of an interdigital surface wave device, which can be adapted for use with one embodiment of the present invention;

FIG. 2 illustrates a cross-sectional view along line A-A of the interdigital surface wave device depicted in FIG. 1, which can be adapted for use with one embodiment of the present invention;

FIG. 3 illustrates a perspective view of an interdigital surface wave device, which can be adapted for use with one embodiment of the present invention;

FIG. 4 illustrates a cross-sectional view along line A-A of the interdigital surface wave device depicted in FIG. 3, which can be adapted for use with one embodiment of the present invention;

FIG. 5 illustrates a block diagram of a wireless surface acoustic wave flow sensor system, which can be implemented in accordance with another embodiment of the present invention;

FIG. 6 illustrates a block diagram of an in-vivo acoustic wave flow sensor system, which can be implemented in accordance with another embodiment of the present invention;

FIG. 7 illustrates a block diagram of an in-vivo acoustic wave flow sensor system, which can be implemented in accordance with an alternative embodiment of the present invention;

FIG. 8 illustrates a block diagram of a wireless surface acoustic wave flow sensor system without a heater, which can be implemented in accordance with an alternative embodiment of the present invention;

FIG. 9 illustrates a block diagram of a cylindrical shape wireless surface acoustic wave flow sensor system, which can be implemented in accordance with an alternative embodiment of the present invention; and

FIG. 10 illustrates a perspective view of a wireless blood flow sensor system, comprising a sensor integrated with a stent for measuring blood flow, in accordance with an embodiment of the present invention;

FIG. 11 illustrates a perspective view of a wireless blood flow sensor system, comprising one or more sensors integrated with a stent for measuring blood flow, in accordance with an alternative embodiment of the present invention;

FIG. 12 illustrates a perspective view of a wireless blood flow sensor system, comprising one or more sensors measuring blood flow, in accordance with an alternative embodiment of the present invention;

FIG. 13 illustrates a perspective view of a wireless blood flow sensor system, comprising an upstream sensor and a downstream sensor integrated with a stent for measuring blood flow, in accordance with an alternative embodiment of the present invention; and

FIG. 14 illustrates a perspective view of an in-line sensor connected to a stent, in accordance with an alternative embodiment of the present invention.

### DETAILED DESCRIPTION OF THE INVENTION

The particular values and configurations discussed in these non-limiting examples can be varied and are cited merely to illustrate at least one embodiment of the present invention and are not intended to limit the scope of the invention.

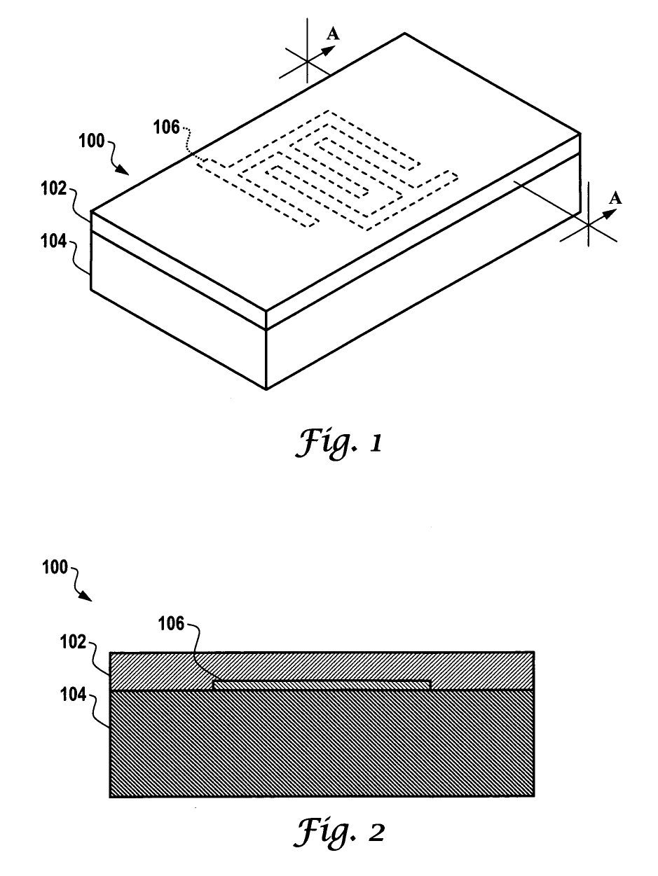

FIG. 1 illustrates a perspective view of an interdigital surface wave device 100, which can be implemented in accordance with one embodiment of the present invention. Surface wave device 100 can be adapted for use in blood flow sensing activities, as described in further detail herein. Surface wave device 100 can be configured to generally include an interdigital transducer 106 formed on a piezoelectric substrate 104. The surface wave device 100 can be implemented in the context of a sensor chip. Interdigital transducer 106 can be configured in the form of an electrode.

FIG. 2 illustrates a cross-sectional view along line A-A of the interdigital surface wave device 100 depicted in FIG. 1, in accordance with one embodiment of the present invention. Piezoelectric substrate 104 can be formed from a variety of substrate materials, such as, for example, quartz, lithium niobate (LiNbO₃), lithium tantalite (LiTaO₃), Li₂B₄O₇, GaPO₄, langasite (La₃Ga₅SiO₁₄), ZnO, and/or epitaxially grown nitrides such as Al, Ga or Zn, to name a few. Interdigital transducer 106 can be formed from materials, which are generally divided into three groups. First, interdigital transducer 106 can be formed from a metal group material (e.g., Al, Pt, Au, Rh, Ir, Cu, Ti, W, Cr, or Ni). Second, interdigital transducer 106 can be formed from alloys such as NiCr or CuAl. Third, interdigital transducer 106 can be formed from metal-nonmetal compounds (e.g., ceramic electrodes based on TiN, CoSi₂, or WC). Depending on the biocompatibility of the substrate and interdigital transducer materials, a thin layer of biocompatible coating 102 may be used to cover the interdigital transducer and the substrate.

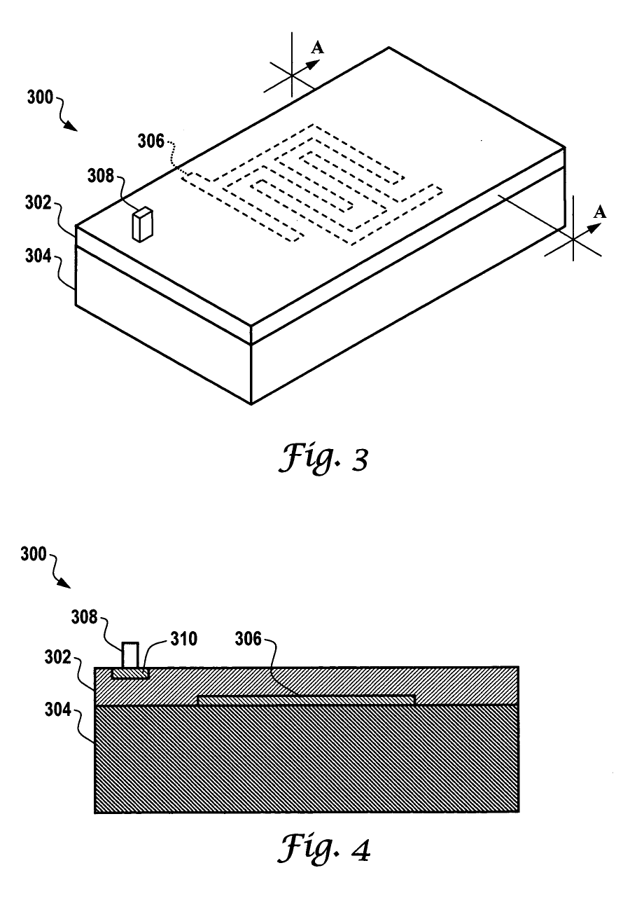

FIG. 3 illustrates a perspective view of an interdigital surface wave device 300, which can be implemented in accordance with an alternative embodiment of the present invention. The configuration depicted in FIGS. 3-4 is similar to that illustrated in FIGS. 1-2, with the addition of an antenna 308, which is connected to and disposed above a wireless excitation component 310 (i.e., shown in FIG. 4). Surface wave device 300 generally includes an interdigital transducer 306 formed on a piezoelectric substrate 304. Surface wave device 300 can therefore function as an interdigital surface wave device, and one, in particular, which utilizing surface-skimming bulk wave techniques. Interdigital transducer 306 can be configured in the form of an electrode. A biocompatible coating 302 can be selected such that there will be no adverse effect to a living body (e.g., human, animal). Various selective coatings can be utilized to implement coating 302.

A change in acoustic properties can be detected and utilized to identify or detect the substance or species absorbed and/or adsorbed by the interdigital transducer 306. Thus, interdigital transducer 306 can be excited via wireless means to implement a surface acoustical model. Thus, antenna 308 and wireless excitation component 310 can be utilized to excite one or more frequency modes associated with the flow of a fluid such as blood for fluid flow analysis thereof.

FIG. 4 illustrates a cross-sectional view along line A-A of the interdigital surface wave device 300 depicted in FIG. 3, in accordance with one embodiment of the present invention. Thus, antenna 308 is shown in FIG. 4 disposed above coating 302 and connected to wireless excitation component 310, which can be formed within an area of coating 302. Similar to the configuration of FIG. 2, Piezoelectric substrate 304 can be formed from a variety of substrate materials, such as, for example, quartz, lithium niobate (LiNbO₃), lithium tantalite (LiTaO₃), Li₂B₄O₇, GaPO₄, langasite (La₃Ga₅SiO₁₄), ZnO, and/or epitaxially grown nitrides such as Al, Ga or Zn, to name a few.

Interdigital transducer 306 can be formed from materials, which are generally divided into three groups. First, interdigital transducer 106 can be formed from a metal group material (e.g., Al, Pt, Au, Rh, Ir, Cu, Ti, W, Cr, or Ni). Second, interdigital transducer 106 can be formed from alloys such as NiCr or CuAl. Third, interdigital transducer 306 can be formed from metal-nonmetal compounds (e.g., ceramic electrodes based on TiN, CoSi₂, or WC).

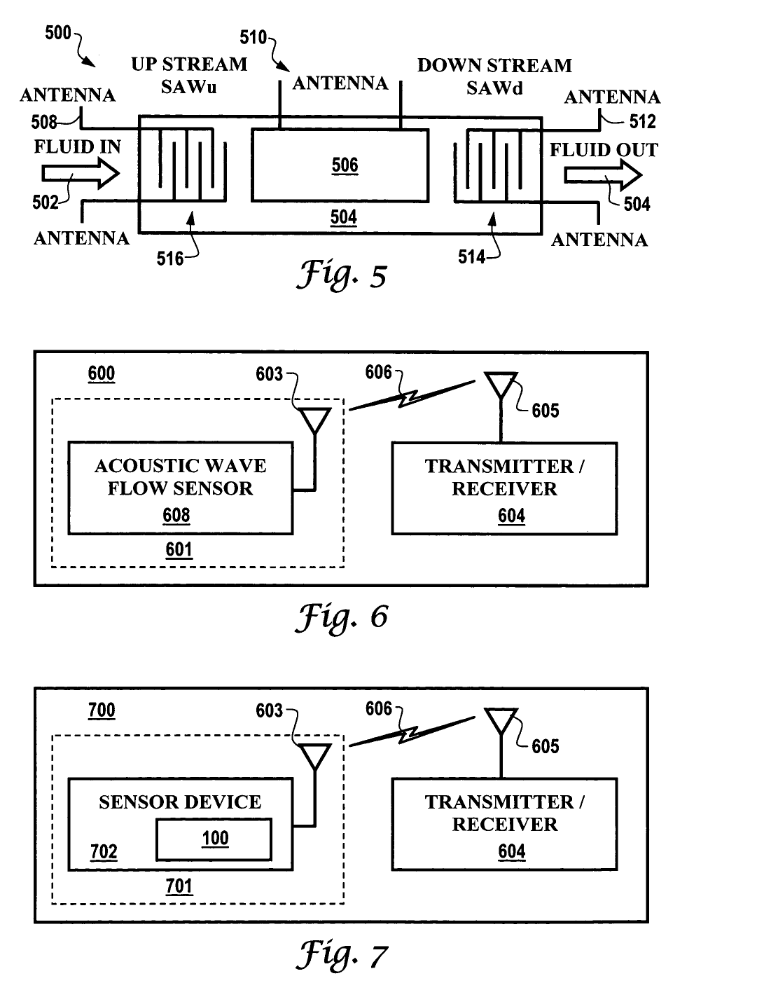

FIG. 5 illustrates a block diagram depicted a perspective view of a wireless SAW flow sensor system 500, which can be implemented in accordance with a preferred embodiment of the present invention. System 500 includes a compartment or structure 504 in which a self-heating heater 506 and an upstream SAWu sensor device 516 can be located. Structure 504 additionally can include a down stream SAWd sensor device 514. Sensor devices 516 and 514 can be implemented as interdigital transducers similar to those depicted in FIGS. 1-4.

Arrows 502 and 504 respectively indicate blood (or other fluid, such as saline) flow in and blood out from compartment or structure 504. An antenna 508 can be integrated with and/or connected to up stream SAWu sensor device 516. System 500 can be, for example, located external to a living body or located within a living body (e.g., within a blood vessel). System 500 can be, for example, implemented within the context of a saline drip device for delivering saline to a living body. Similarly, a second antenna 512 can be integrated with and/or connected to SAWd down stream sensor device 514. Additionally, a third antenna 510 can be integrated with and/or connected to self-heating heater 506. Note that self-heating heater 506 can be powered by converting RF power to heat.

The self-heating heater 506 can absorbs energy from RF power and convert it to heat. This self-heating portion can be formed from acoustically “lossy” materials, or acoustical absorber, in which the dissipation of acoustic energy in such material causes heating of the substrate. For a given thermal conductivity and effective thermal mass of the substrate, the quiescent surface temperature can eventually achieve steady state. Self-heating heater 506 can also be configured from a resistor-heater type material.

FIG. 6 illustrates a block diagram of an in-vivo acoustic wave flow sensor system 600, which can be implemented in accordance with a preferred embodiment of the present invention. System 600 generally includes an acoustic wave flow sensor device 608, which can be implemented in a configuration similar to that of sensor system 500 depicted in FIG. 5. For example, acoustic wave flow sensor device 608 can be equipped with one or more digital transducers, such as those depicted in FIG. 5.

Device 608 can be configured to include an acoustic coating such as that depicted in FIG. 1. Acoustic wave flow sensor device 608 can be coupled to and/or integrated with an antenna 603. Antenna 603 can receive and/or transmit data to and from a transmitter/receiver 604. In general, the antenna 603 can be connected to device 608, such that antenna 605 receives one or more signals, which can excite an acoustic device thereof to produce a frequency output associated with the flow of blood for analysis thereof.

Note that acoustic wave flow sensor device 608 can be associated with a microprocessor (i.e., not shown in FIG. 6), which can process and control data for controlling one or more sensing functions of acoustic wave flow sensor device 608. An example of a microprocessor that can be adapted for use with the embodiments disclosed herein include a central processing unit (CPU) or other similar device, such as those found in personal computers, personal digital assistant (PDA) and other electronic devices. Such a microprocessor can control logical operations associated with, for example, acoustic wave flow sensor device 608. Such a microprocessor can be integrated with acoustic wave flow sensor device 608 or located separately from device 608, while still controlling and processing data associated with sensing functions thereof, depending upon design considerations.

Acoustic wave flow sensor device 608 and antenna 603 together can form a passive, wireless, in vivo acoustic wave flow sensor device 601, which can be implanted within a human being. Wireless interrogation, as represented by arrow 606 can provide the power and data collection necessary for the proper functioning of device 601. Device 601 can be implemented via a variety of surface acoustic wave technologies, such as Rayleigh waves, shear horizontal waves, love waves, and so forth.

FIG. 7 illustrates a block diagram of an in-vivo acoustic wave flow sensor system 700, which can be implemented in accordance with an alternative embodiment of the present invention. Note that in FIGS. 6 and 7, identical parts or elements are generally indicated by identical reference numerals. System 700 is therefore similar to system 600 depicted in FIG. 6, but includes some slight modifications. For example, a sensor device 702 is utilized in place of device 520. Sensor device 702 incorporates device 100 depicted in FIG. 1. Thus, sensor device 702 and transmitter/receiver 602 together form a sensing device 701, which can be utilized to monitor liquid flow rate, such as, for example, that of human blood flowing within a human body.

Note that as utilized herein the terms “transmitter/receiver” and “transmitter and receiver unit” can be utilized interchangeably and can also refer to an integrated unit that comprises both a transmitter and receiver, or to separate transmitters and receivers, which may be located remotely from one another. Additionally, the terms “transmitter unit” and “transmitter” can be utilized interchangeably to refer the same device. The terms “receiver unit” and “receiver” can also be utilized interchangeably to refer to the same device. The transmitter and/or receiver can thus transmit short range radio frequency energy field(s) to one or more antennae associated with said sensor, such that the transmitter and the receiver can receive data transmitted from the sensor via one or more antennae.

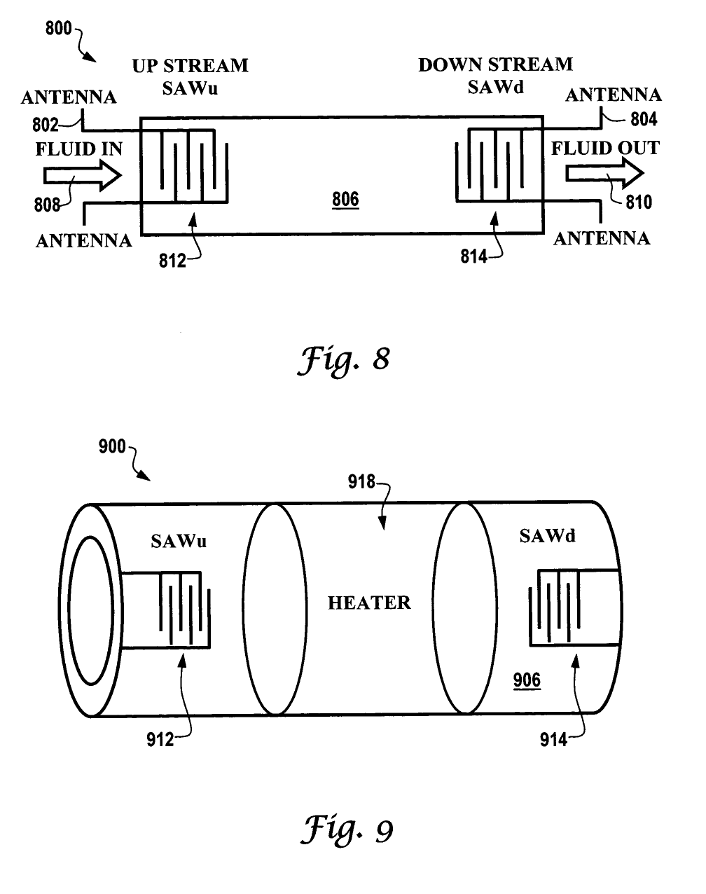

FIG. 8 illustrates a block diagram of a wireless surface acoustic wave flow sensor system 800, which can be implemented without a heater, in accordance with an alternative embodiment of the present invention. System 800 generally includes a compartment or structure 806 in which an upstream SAWu sensor device 812 (i.e., a sensor) can be located. Structure 806 additionally can include a down stream SAWd sensor device 814 (i.e., as sensor). Note that the term “sensor device” and “sensor” as utilized herein can be utilized interchangeably to refer to the same feature. Sensor devices 812 and 814 can be implemented, for example, as interdigital transducers similar to those depicted in FIGS. 1-4. Structure 806 can be implemented as or integrated with a stent.

Arrows 808 and 810 respectively indicate fluid or blood flow in out of compartment or structure 806. An antenna 802 can be integrated with and/or connected to up stream SAWu sensor device 812. Similarly, a second antenna 814 can be integrated with and/or connected to SAWd down stream sensor device 814. Note that the antennas such as antenna 802 and the other antennas discussed herein can be utilized for a variety of purposes. For example, one antenna can be utilized to receive excitation signals, while the other antenna can be utilized to transmit results.

FIG. 9 illustrates a block diagram of a cylindrical shape wireless surface acoustic wave flow sensor system 900, which can be implemented in accordance with an alternative embodiment of the present invention. System 900 includes a cylindrical-shaped compartment or structure 906 in which a self-heating heater 918 and an upstream SAWu sensor device 912 can be located. Structure 906 additionally can include a down stream SAWd sensor device 914. Sensor devices 912 and 914 can be, for example, implemented as interdigital transducers similar to those depicted in FIGS. 1-4.

The SAWu sensor device 912, heater 918 and SAWd sensor device 914 can be located on the inside wall of structure 906 with respective connections at the ends thereof. In the configuration of system 900, 350 degrees of the inside circumference can be utilized for the heater resistor or heater 918, which leaves sufficient space for configuring all connects at the edges of structure 906. Structure 906 can comprise, for example, a stent used in medical procedures. System 900 can be implemented in the context of a stent. Heater 918 can, for example, be integrated into the walls of the stent (e.g., structure 906) to permit a small amount of heating of blood flowing through structure 906 (i.e., a stent). The blood can be heated by heater 918 a few degrees above ambient.

In terms of coating selection, biocompatibility involves the acceptance of an artificial implant by the surrounding tissue and by the body as a whole. Biocompatible materials do not irritate the surrounding structures, do not provoke an abnormal inflammatory response, do not incite allergic reactions, and do not cause cancer.

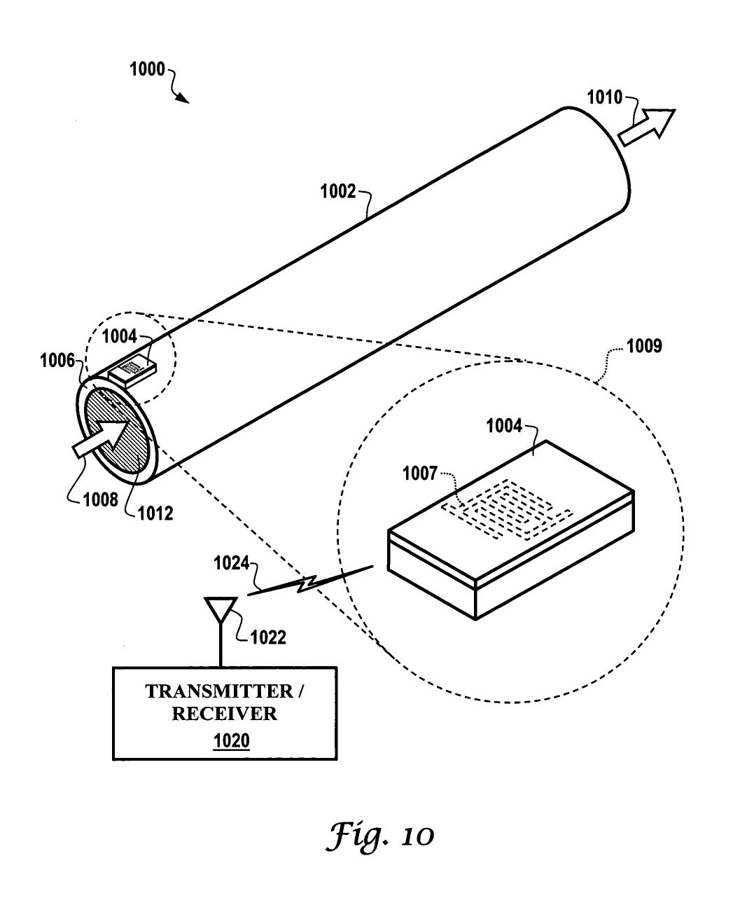

FIG. 10 illustrates a perspective view of a wireless blood flow sensor system 1000, comprising a sensor 1004 integrated with a stent 1002 for measuring blood flow, in accordance with one embodiment of the present invention. Stent 1002 comprises a cylindrical-shaped structure that includes a continuous cylindrical shaped wall (or walls) 1006. Sensor 1004 can be integrated into walls 1006 of stent 1002. Arrows 1008 and 1010 respectively represent the flow of blood through stent 1002 when stent 1002 is located within a blood vessel.

Stent 1002 further includes a cylindrically shaped internal gap 1012 through which blood flows through stent 1002, as indicated by arrows 1008 and 1010. Sensor 1004 can comprise, for example, a device that includes one or more antennas and a sensor component or sensor device such as an interdigital transducer. Sensor 1004 is generally analogous to, for example, upstream SAWu sensor device 812 or downstream SAWu sensor device 814 depicted in FIG. 8.

As indicated in FIG. 10 by a dashed circle 1009, which represents an enhanced view of sensor 1002, an antenna 1007, such as, for example, antenna 802 and/or antenna 804 depicted in FIG. 8, can be integrated with or connected to sensor 1004. Additionally, system 1000 can include a transmitter/receiver 1020 which is connected to an antenna 1022. Antenna 1007 of sensor 1004 can receive and/or transmit data to and from transmitter/receiver 1020.

In general, antenna 1007 of sensor 1004 is analogous to antenna 506 of FIG. 5, antenna 603 of FIGS. 6-7 and/or antennas 802 and 804 of FIG. 8. Antenna 1022 of transmitter/receiver 1020 (i.e., a transmitter and receiver unit) can transmit one or more signals to sensor 1004, which can excite sensor 1004 to produce a frequency output associated with the flow of blood through stent 1002 for analysis thereof. Note that in FIGS. 10-13, similar or identical parts, components or elements are generally indicated by identical reference numerals. Thus, FIGS. 11-13 represent variations to the embodiment of system 1000 disclosed in FIG. 10.

FIG. 11 illustrates a perspective view of a wireless blood flow sensor system 1100, comprising one or more sensors 1004 and 1005 integrated with stent 1002 for measuring blood flow, in accordance with an alternative embodiment of the present invention. System 1100 of FIG. 11 is thus similar to system 1000 of FIG. 10, with the exception that a plurality of sensors 1004 and 1005 can be integrated into the walls 1006 of stent 1002. Note that sensor 1004 and 1005 can be implemented as identical sensors, which are structurally identical to one another. Thus, sensor 1005 can include an antenna similar to that of 1007 depicted in FIG. 10.

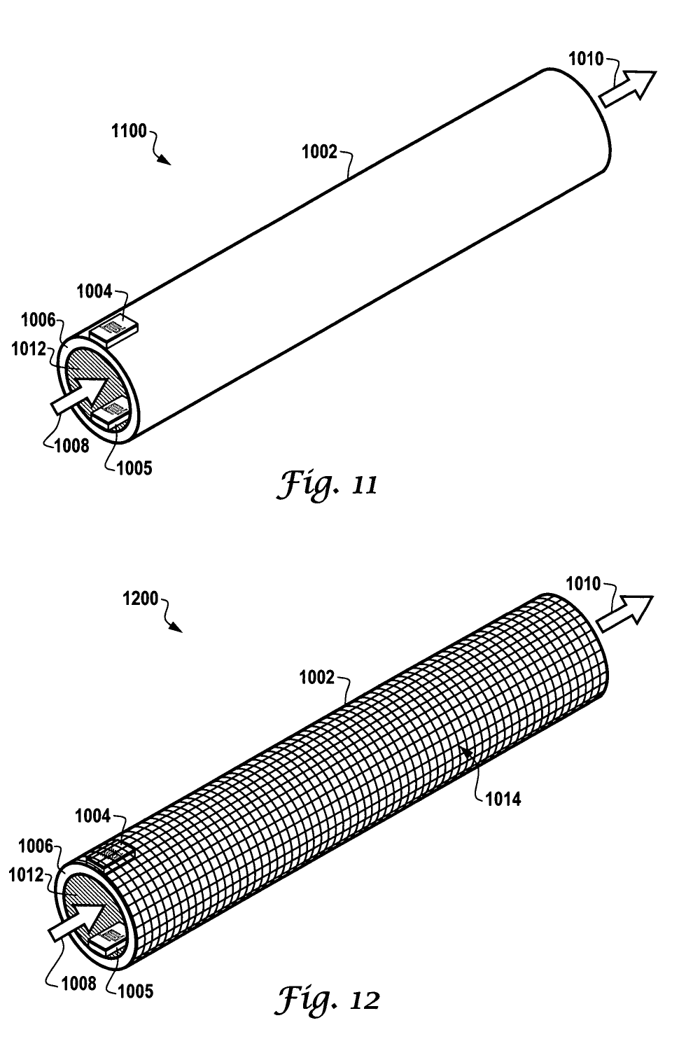

FIG. 12 illustrates a perspective view of a wireless blood flow sensor system 1200, comprising one or more sensors 1004 and 1005 for measuring blood flow, in accordance with an alternative embodiment of the present invention. System 1200 of FIG. 12 is thus similar to system 1100 of FIG. 11 and system 1000 of FIG. 10, but differs in the addition of a wire mesh 1014 integrated with stent 1002. The stent wire mesh can not only structurally support stent 1002, but may support the functions of antennas such as, 1007 of sensor 1004 and antennas associated with sensor 1005. Additionally, wire mesh 1014 can support the function of the antenna 1022 of the transmitter/receiver 1020 depicted in FIG. 10.

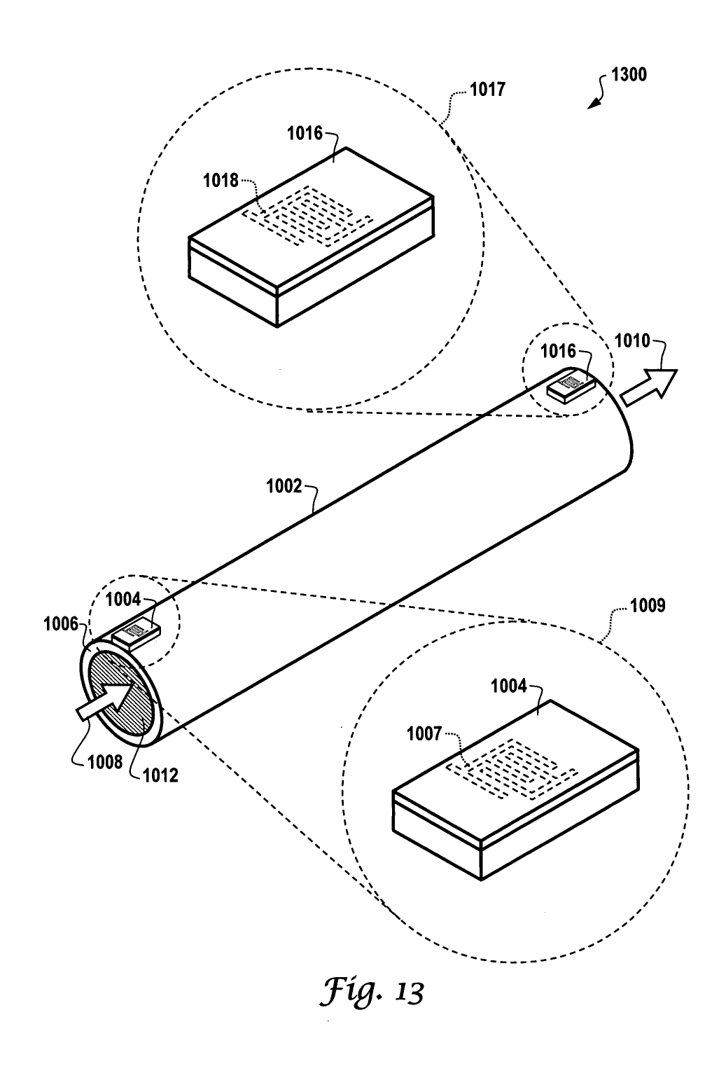

FIG. 13 illustrates a perspective view of a wireless blood flow sensor system 1300, comprising an upstream sensor 1004 and a downstream sensor 1016 integrated with a stent 1002 for measuring blood flow, in accordance with an alternative embodiment of the present invention. Upstream sensor 1004 can be implemented as a sensor device, such as, for example, upstream SAWu sensor device 812 depicted in FIG. 8. Downstream sensor 1016 can be implemented as a sensor device, such as, for example, downstream sensor 814 depicted in FIG. 8. Dashed circle 1017 indicates that upstream sensor 1016 is structurally similar to that of downstream sensor 1004 in that upstream sensor 1016 includes an antenna 1018 similar to that of antenna 1007. Antennas 1007 and 1018 can be implemented similar to that of antenna 308 depicted in FIG. 3.

Additionally sensors 1007 and 1016 can function similar to that of surface wave device 309 of FIG. 3, such that each antenna 1007 and 1018 is connected to and disposed above a wireless excitation component similar to that of wireless excitation component 310 depicted in FIG. 4. Sensors 1006 and 1016 can be configured to include an interdigital transducer (e.g., interdigital transducer 306 of FIGS. 3-4) formed on a piezoelectric substrate 304. Surface wave device 300 can therefore function as an interdigital surface wave device, and one, in particular, which utilizing surface-skimming bulk wave techniques. Interdigital transducer 306 can be configured in the form of an electrode. A biocompatible coating 302 can be selected such that there will be no adverse effect to the human body. Various selective coatings can be utilized to implement coating 302.

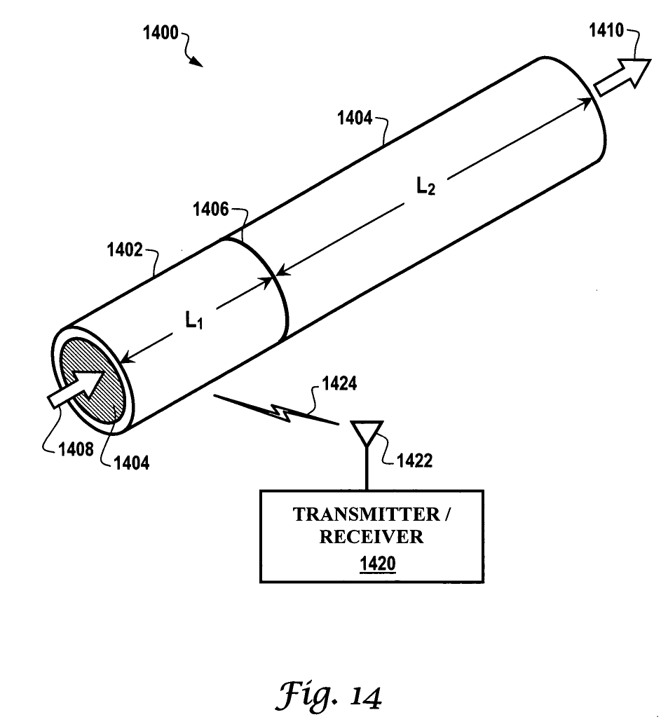

FIG. 14 illustrates a perspective view of an in-line sensor 1402 connected to a stent 1404, in accordance with an alternative embodiment of the present invention. Sensor 1402 can function not only as a flow sensor, such as flow sensor 1004, but also as a temperature and/or pressure sensor. Thus, sensor 1402 can be located in series or “in-line” with stent 1404, and can be, for example approximately half the length of stent 1404. The length of sensor 1402 is indicated by L₁, while the length of stent 1404 is indicated by L₂ such that L₁=½ L₂. Sensor 1402 includes a cylindrical gap 1404 through which blood and/or fluid can flow, as indicated by arrows 1408 and 1410.

Sensor 1402 is generally connected to stent 1404 at interface 1406. The connection between sensor 1402 and stent 1404 can be implemented, for example, via an interlocking mechanism. Sensor 1402 butts up against stent 1404 such that sensor 1402 and stent 1404 have the same inner diameter and outer diameter dimensions. Sensor 1402 can be configured to include one or more microstructure temperature sensing elements formed on a substrate within a hermetically sealed area thereof. Sensor 1402 can be equipped with an antenna similar to that, for example, of antennas 1007 and/or 1018 in order to communicate with transmitter/receiver 1420. Thus, in addition to providing blood flow data, sensor 1402 can also provide pressure and/or temperature data.

The microstructure temperature-sensing elements of sensor 1402 can be implemented, for example, as SAW (surface acoustic wave) temperature-sensing elements. Sensor 1402 can be, for example, a cylindrically shaped Interdigital Transducer (IDT). Additionally, one or more microstructure pressure-sensing elements can be implemented on or above a sensor diaphragm (not shown in FIG. 14) on a substrate from which sensor 1402 is formed.

The embodiments and examples set forth herein are presented to best explain the present invention and its practical application and to thereby enable those skilled in the art to make and utilize the invention. Those skilled in the art, however, will recognize that the foregoing description and examples have been presented for the purpose of illustration and example only. Other variations and modifications of the present invention will be apparent to those of skill in the art, and it is the intent of the appended claims that such variations and modifications be covered.

The description as set forth is not intended to be exhaustive or to limit the scope of the invention. Many modifications and variations are possible in light of the above teaching without departing from the scope of the following claims. It is contemplated that the use of the present invention can involve components having different characteristics. It is intended that the scope of the present invention be defined by the claims appended hereto, giving full cognizance to equivalents in all respects.

1. A blood flow sensing system, comprising:
a sensor coupled to at least one antennae, wherein said sensor measures a flow of blood within a blood vessel when stimulated with a short range energy field detectable by said at least one antennae;
a transmitter and a receiver, wherein said transmitter and said receiver can transmit said short range energy field to said at least one antennae of said sensor, wherein said receiver can receive data transmitted from said sensor via said at least one antennae; and
a stent integrated with sensor, wherein said stent comprises a small diameter cylinder that props open a blood vessel and wherein said stent is moveable into said blood vessel to form a rigid support for holding said blood vessel open.

2. The system of claim 1 wherein said stent comprises a metal structure forming at least one part of said small diameter cylinder, wherein said metal structure supports a functionality of said at least one antennae.

3. The system of claim 2 wherein said metal structure comprises at least one of the following: a wire mesh or a wire spiral.

4. The system of claim 1 wherein said sensor measures heat transfer to blood within said blood vessel.

5. The system of claim 1 wherein said stent comprises an arterial stent and wherein said blood vessel comprises an artery.

6. The system of claim 1 wherein:
said sensor comprises a surface acoustic wave flow sensor comprising at least one interdigital transducer and a self-heating heater formed upon a piezoelectric substrate, wherein said interdigital transducer is selected to introduce negligible electrical coupling to surface waves thereof; and
wherein said at least one said antennae is connected to said at least one interdigital transducer, wherein said antenna receives at least one signal, which excites said at least one interdigital transducer to produce a frequency output associated with said flow of blood for analysis thereof.

7. The system of claim 6 wherein said transmitter and said receiver are located external to a living body associated with said blood vessel.

8. The system of claim 7 wherein said surface acoustic wave flow sensor generates surface acoustic wave resonation delta frequency data that is receivable by said receiver.

9. The system of claim 6 wherein said surface acoustic wave flow sensor comprises a closed loop delay line that shifts based on upstream and downstream temperature changes associated with said flow of blood.

10. The system of claim 1 further comprising:
at least one radiating resonant circuit integrated with said sensor, wherein said at least one radiating resonant circuit comprises at least one upstream sensor resistor and at least one downstream sense resistor; and
a cylindrical structure within which said sensor is located, such that said at least one upstream sense resistor and said at least one downstream sense resistor are integrated into a wall of said cylindrical structure in order to heat said flow of blood above an ambient temperature thereof.

11. The system of claim 6 wherein said at least one frequency output comprise at least one of the following types of data:
flexural plate mode (FPM) data, acoustic plate mode data;
shear-horizontal acoustic plate mode (SH-APM) data;
amplitude plate mode (APM) data;
thickness shear mode (TSM) data;
surface acoustic wave mode (SAW), and bulk acoustic wave mode (BAW) data;
torsional mode data;
love wave data;
leaky surface acoustic wave mode (LSAW) data;
pseudo surface acoustic wave mode (PSAW) data;
transverse mode data, surface-skimming mode data;
surface transverse mode data;
harmonic mode data; and
overtone mode data.

12. A blood flow sensing system, comprising:
a sensor coupled to at least one antennae, wherein said sensor measures a flow of blood within a blood vessel when stimulated with a short range energy field detectable by said at least one antennae and wherein said sensor measures heat transfer to blood within said blood vessel;
a transmitter and a receiver which transmit said short range energy field to said at least one said antennae coupled to said sensor, wherein said receiver receives data transmitted from said sensor via said at least one antennae; and
a stent integrated with sensor, wherein said stent comprises a small diameter cylinder that props open a blood vessel, wherein said stent is moveable into said blood vessel to form a rigid support for holding said blood vessel open and wherein said stent comprises a metal structure that supports a functionality of said at least one antennae.

13. The system of claim 12 wherein:
said sensor comprises a surface acoustic wave flow sensor comprising at least one interdigital transducer and a self-heating heater formed upon a piezoelectric substrate, wherein said interdigital transducer is selected to introduce negligible electrical coupling to surface waves thereof; and
wherein said at least one antennae is connected to said at least one interdigital transducer, wherein said antenna receives at least one signal, which excites said at least one interdigital transducer to produce a frequency output associated with said flow of blood for analysis thereof.

14. The system of claim 13 wherein said transmitter and receiver unit is located external to a living body associated with said blood vessel.

15. The system of claim 13 wherein said sensor further comprises at least one interdigital transducer for measuring pressure.

16. The system of claim 13 wherein said sensor further comprises at least one interdigital transducer for measuring temperature.

17. A blood flow sensing system, comprising:
a sensor coupled to at least one antennae, wherein said sensor measures a flow of blood within a blood vessel when stimulated with a short range energy field detectable by said at least one antennae and wherein said sensor measures heat transfer to blood within said blood vessel;
at least one temperature sensing element integrated with said sensor;
at least one pressure sensing element integrated with said sensor;
a transmitter and a receiver which transmit said short range energy field to said at least one antennae of said sensor, wherein said transmitter and receiver unit also receives data transmitted from said sensor via said at least one antennae; and
a stent integrated with sensor, wherein said stent comprises a small diameter cylinder that props open a blood vessel and wherein said stent is moveable into said blood vessel to form a rigid support for holding said blood vessel open and wherein said stent comprises a metal structure that supports a functionality of said at least one antennae, wherein said sensor is capable of measuring said flow of said blood within said blood vessel.

18. The system of claim 17 wherein said at least one temperature sensing element integrated with said sensor comprises an interdigital transducer and measures temperature within said blood vessel.

19. The system of claim 17 wherein said at least one pressure sensing element integrated with said sensor comprises an interdigital transducer and measures pressure within said blood vessel.

20. The system of claim 17 wherein said sensor comprises a surface acoustic wave flow sensor that generates surface acoustic wave resonation delta frequency data receivable by said transmitter and receiver unit.

21. The system of claim 17 further comprising:
at least one radiating resonant circuit integrated with said sensor, wherein said at least one radiating resonant circuit comprises at least one upstream sensor resistor and at least one downstream sense resistor; and
a cylindrical structure within which said sensor is located, such that said at least one upstream sense resistor and said at least one downstream sense resistor are integrated into a wall of said cylindrical structure in order to heat said flow of blood above an ambient temperature thereof.

22. The system of claim 1 wherein said transmitter comprises a data transmission function for modifying a behavior of said sensor.

23. The system of claim 1 further comprising a microprocessor associated with said sensor, wherein said microprocessor processes and controls data for controlling at least one sensing function of said sensor.

24. The system of claim 1 further comprising a microprocessor operable to control the sensing functions.

25. A fluid flow sensing system, comprising:
a sensor coupled to at least one antennae, wherein said sensor measures a flow of fluid when stimulated with a short range energy field detectable by said at least one antennae;
a transmitter and a receiver, wherein said transmitter and said receiver can transmit said short range energy field to said at least one antennae of said sensor, wherein said receiver can receive data transmitted from said sensor via said at least one antennae; and
a tubular structure within which said sensor is located, wherein said sensor measures said flow of fluid within said tubular structure.

26. The system of claim 25 wherein said flow of fluid comprises a blood flow and wherein said tubular structure is configured such that a flow of blood is increased within a blood vessel as a result of said tubular structure being located within said blood vessel.
